(19) United States
(12) Patent Application Publication
(10) Pub. No.: US 2017/0354347 A1
(43) Pub. Date: Dec. 14, 2017

(54) POSITION DETECTION SYSTEM AND CAPSULE MEDICAL APPARATUS GUIDANCE SYSTEM

(71) Applicant: OLYMPUS CORPORATION, Tokyo (JP)

(72) Inventor: Atsushi CHIBA, Tokyo (JP)

(73) Assignee: OLYMPUS CORPORATION, Tokyo (JP)

(21) Appl. No.: 15/666,612

(22) Filed: Aug. 2, 2017

Related U.S. Application Data
(63) Continuation of application No. PCT/JP2015/079886, filed on Oct. 22, 2015.

(30) Foreign Application Priority Data
Feb. 18, 2015 (JP) 2015-030002

(51) Int. Cl. A61B 5/06; A61B 1/04; A61B 1/00

(52) U.S. Cl. CPC A61B 5/062; A61B 1/00158; A61B 1/041

(57) ABSTRACT

A position detection system includes: a cylindrical detection coil configured to detect a magnetic field generated by a magnetic field generation unit; and a calculation unit configured to calculate at least one of a position and a direction of the magnetic field generation unit based on the magnetic field detected by the detection coil. A relationship between a diameter Ds and a length Ls in a winding direction, of the detection coil, satisfies Formula (1), and each of coefficients G₁, G₂, and G₃ in Formula (1) is respectively given by each of Formulae (2), (3) and (4).

$$\begin{matrix}\vert {G}_{1} {( \frac{{L}_{S}}{{D}_{S}} )}^{2} + {G}_{2} ( \frac{{L}_{S}}{{D}_{S}} ) + {G}_{3} \vert ≦ 0.2 & ( 1 ) \\ {G}_{1} = - 1.73 {10}^{- 5} {D}_{S}^{2} + 7.36 {10}^{- 3} {D}_{S} - 4.71 {10}^{- 2} & ( 2 ) \\ {G}_{2} = 3.74 {10}^{- 5} {D}_{S}^{2} - 1.54 {10}^{- 3} {D}_{S} + 1.16 {10}^{- 2} & ( 3 ) \\ {G}_{3} = - 8.96 {10}^{- 5} {D}_{S}^{2} - 1.74 {10}^{- 3} {D}_{S} + 1.30 {10}^{- 2} & ( 4 )\end{matrix}$$

### CROSS-REFERENCE TO RELATED APPLICATION

This application is a continuation of PCT international application Ser. No. PCT/JP2015/079886 filed on Oct. 22, 2015 which designates the United States, incorporated herein by reference, and which claims the benefit of priority from Japanese Patent Applications No. 2015-030002, filed on Feb. 18, 2015, incorporated herein by reference.

### BACKGROUND

### 1. Technical Field

The disclosure relates to a position detection system configured to detect a position or a direction of a capsule medical apparatus by detecting, outside a subject, a magnetic field that is generated from the capsule medical apparatus introduced into the subject and relates to a capsule medical apparatus guidance system configured to guide the capsule medical apparatus.

### 2. Related Art

Conventionally, a capsule medical apparatus configured to be introduced into a subject and obtain various types of information on the subject, or configured to administer a drug, or the like, to the subject, has been developed. A known example of this is a capsule endoscope formed into a size that can be introduced into the gastrointestinal tract (lumen) of the subject. The capsule endoscope has an imaging function and wireless communication function inside a capsule-shaped casing. The capsule endoscope is swallowed by the subject and thereafter captures images while moving inside the gastrointestinal tract, and wirelessly transmits image data of the image of an internal portion of an organ of the subject (hereinafter, also referred to as an in-vivo image) in sequence.

A system for detecting the position and the direction of such a capsule medical apparatus inside the subject has been developed. For example, JP 2008-132047 A discloses a position detection system configured to provide a coil that generates a magnetic field by receiving power (hereinafter, referred to as a magnetic field generation coil) within the capsule medical apparatus, detect a magnetic field generated from this magnetic field generation coil, using a plurality of magnetic field detection coils (hereinafter, referred to as a detection coil) provided outside the subject, and perform position detection calculation of the capsule medical apparatus on the basis of the intensity of the detected magnetic field.

### SUMMARY

In some embodiments, a position detection system includes: a cylindrical detection coil configured to detect a magnetic field generated by a magnetic field generation unit; and a calculation unit configured to calculate at least one of a position and a direction of the magnetic field generation unit based on the magnetic field detected by the detection coil. A relationship between a diameter Ds and a length Ls in a winding direction, of the detection coil, satisfies Formula (1), and each of coefficients G₁, G₂, and G₃ in Formula (1) is respectively given by each of Formulae (2), (3) and (4).

$$\begin{matrix}\vert {G}_{1} {( \frac{{L}_{S}}{{D}_{S}} )}^{2} + {G}_{2} ( \frac{{L}_{S}}{{D}_{S}} ) + {G}_{3} \vert ≦ 0.2 & ( 1 ) \\ {G}_{1} = - 1.73 {10}^{- 5} {D}_{S}^{2} + 7.36 {10}^{- 3} {D}_{S} - 4.71 {10}^{- 2} & ( 2 ) \\ {G}_{2} = 3.74 {10}^{- 5} {D}_{S}^{2} - 1.54 {10}^{- 3} {D}_{S} + 1.16 {10}^{- 2} & ( 3 ) \\ {G}_{3} = - 8.96 {10}^{- 5} {D}_{S}^{2} - 1.74 {10}^{- 3} {D}_{S} + 1.30 {10}^{- 2} & ( 4 )\end{matrix}$$

In some embodiments, a capsule medical apparatus guidance system includes the above-described the position detection system. The capsule medical apparatus further incorporates a magnet. The capsule medical apparatus guidance system further includes a guidance magnetic field generation apparatus configured to generate a magnetic field for guiding the capsule medical apparatus by causing the magnet to act.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a position detection system according to a first embodiment of the disclosure;

FIG. 2 is a schematic diagram illustrating an exemplary internal structure of a capsule endoscope illustrated in FIG. 1;

FIG. 3 is a schematic diagram illustrating a shape of a detection coil illustrated in FIG. 1;

FIG. 4 is a plan view illustrating exemplary arrangement of the detection coil on a panel of a magnetic field detection apparatus illustrated in FIG. 1;

FIG. 5 is a plan view illustrating another exemplary arrangement of the detection coil on the panel of the magnetic field detection apparatus illustrated in FIG. 1;

FIG. 6A is a schematic diagram illustrating a result of simulation for obtaining a detected magnetic field error (Dₛ=10 mm, Lₛ=30 mm);

FIG. 6B is a schematic diagram illustrating a result of simulation for obtaining a detected magnetic field error (Dₛ=10 mm, Lₛ=25 mm);

FIG. 6C is a schematic diagram illustrating a result of simulation for obtaining a detected magnetic field error (Dₛ=10 mm, Lₛ=20 mm);

FIG. 6D is a schematic diagram illustrating a result of simulation for obtaining a detected magnetic field error (Dₛ=10 mm, Lₛ=15 mm);

FIG. 6E is a schematic diagram illustrating a result of simulation for obtaining a detected magnetic field error (Dₛ=10 mm, Lₛ=10 mm);

FIG. 6F is a schematic diagram illustrating a result of simulation for obtaining a detected magnetic field error (Dₛ=10 mm, Lₛ=5 mm);

FIG. 7 is a graph illustrating correlation between a ratio of length/diameter of the detection coil and the detected magnetic field error;

FIG. 8 is a schematic diagram illustrating an exemplary configuration of a capsule medical apparatus guidance system according to a second embodiment of the disclosure;

FIG. 9 is a schematic diagram illustrating an exemplary internal structure of a capsule endoscope illustrated in FIG. 8; and

FIG. 10 is a schematic diagram illustrating an exemplary configuration of a guidance magnetic field generation apparatus illustrated in FIG. 8.

### DETAILED DESCRIPTION

A position detection system according to embodiments of the disclosure will be described with reference to the drawings. The following description will exemplify a capsule endoscope configured to be introduced into the subject orally and to capture an image of an internal portion (lumen) of the subject as one mode of a capsule medical apparatus as a detection target by the position detection system according to the present embodiment. The disclosure, however, is not limited to this embodiment. In other words, the disclosure is applicable to position detection for various capsule-shaped medical apparatuses such as a capsule endoscope that moves inside the lumen from the esophagus to the anus of the subject, a capsule medical apparatus that delivers a drug, or the like, to internal portions of the subject, and a capsule medical apparatus including a pH sensor for measuring pH within the subject.

Note that the drawings in the following description merely schematically illustrate the shapes, sizes, and positional relations to such degrees that the contents of the disclosure are understandable. Accordingly, the disclosure is not limited only to the shapes, sizes, and positional relations exemplified in the individual drawings. In the drawings, same reference signs are attached to the same portions.

### First Embodiment

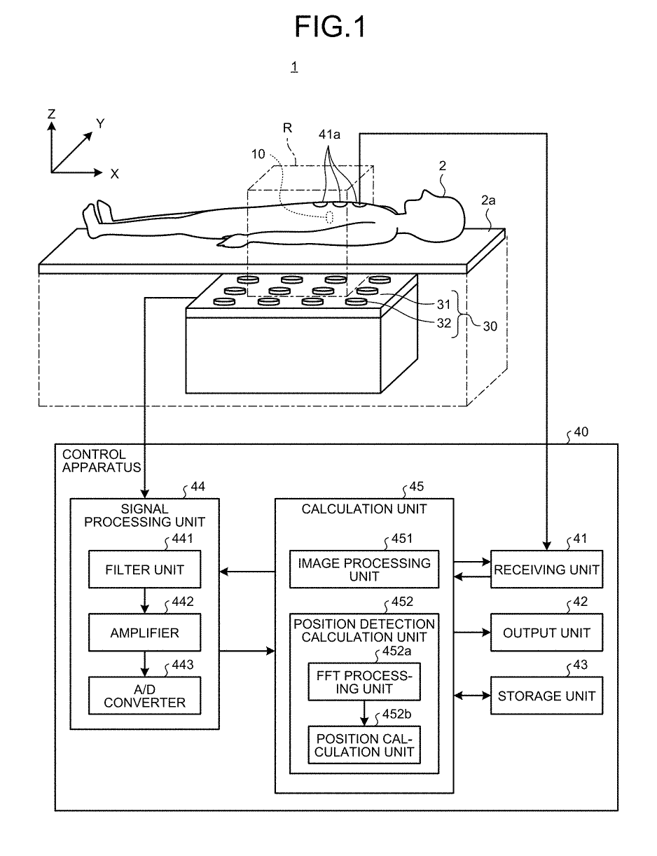

FIG. 1 is a schematic diagram illustrating a position detection system according to a first embodiment of the disclosure. As illustrated in FIG. 1, a position detection system 1 according to the first embodiment includes a capsule endoscope 10, a magnetic field detection apparatus 30, and a control apparatus 40. The capsule endoscope 10, as an exemplary capsule medical apparatus introduced into a lumen of a subject 2, transmits image data obtained by capturing the inside of a subject 2, by superposing the data onto radio signals. The magnetic field detection apparatus 30 is provided below a bed 2a on which the subject 2 is placed and detects an alternating magnetic field generated by the capsule endoscope 10. The control apparatus 40 detects at least any of the position of the capsule endoscope 10 and the direction (posture) of the capsule endoscope 10 on the basis of the alternating magnetic field detected by the magnetic field detection apparatus 30.

Hereinafter, an upper surface of the bed 2a, that is, a placement surface for the subject 2 is defined as an X-Y plane (horizontal plane), and a direction orthogonal to the X-Y plane is defined as a Z-direction (vertical direction).

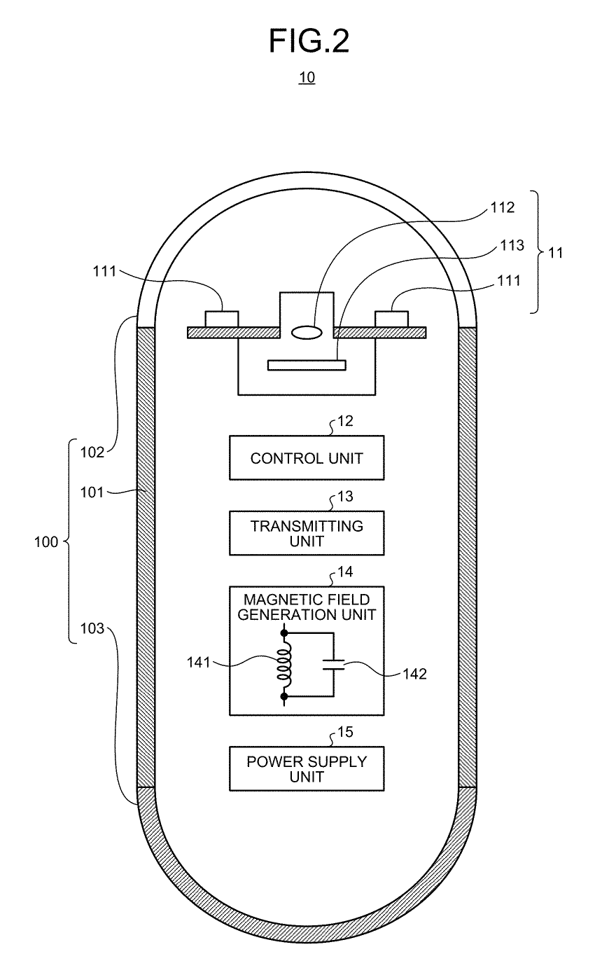

FIG. 2 is a schematic diagram illustrating an exemplary internal structure of the capsule endoscope 10 illustrated in FIG. 1. As illustrated in FIG. 2, the capsule endoscope 10 includes a casing 100, an imaging unit 11, a control unit 12, a transmitting unit 13, a magnetic field generation unit 14, and a power supply unit 15. The casing 100 is a capsule-shaped casing formed into a size that can easily be introduced into the lumen of the subject 2. The imaging unit 11 is contained in the casing 100 and captures the inside of the subject 2 to obtain an imaging signal. The control unit 12 controls operation of each of components, including the imaging unit 11, of the capsule endoscope 10 and performs predetermined signal processing on the imaging signal obtained by the imaging unit 11. The transmitting unit 13 wirelessly transmits the signal-processed image signal. The magnetic field generation unit 14 generates an alternating magnetic field for position detection of the capsule endoscope 10. The power supply unit 15 supplies power to each of the components of the capsule endoscope 10.

The casing 100 is an outer casing formed into a size that can be introduced into the inside of the organ of the subject 2. The casing 100 includes a cylindrical casing 101 having a cylindrical shape, and dome-shaped casings 102 and 103 each having dorm-like shapes, and is formed with both opening ends of the cylindrical casing 101 being closed by the dome-shaped casings 102 and 103. The cylindrical casing 101 is formed of a colored member substantially opaque for the visible light. At least one of the dome-shaped casings 102 and 103 (the dome-shaped casing 102 on a side of the imaging unit 11 in FIG. 2) is formed of an optical member transparent for the light having a predetermined wavelength band, such as visible light. Note that, while one imaging unit 11 is provided on one side, namely, on the side of the dome-shaped casing 102 in FIG. 2, it is allowable to provide two imaging units 11. In this case, also the dome-shaped casing 103 is formed of a transparent optical member. The casing 100 configured in this manner contains, using fluid-tight sealing, the imaging unit 11, the control unit 12, the transmitting unit 13, the magnetic field generation unit 14, and the power supply unit 15.

The imaging unit 11 includes an illumination unit 111 such as an LED, an optical system 112 such as a condenser lens, and an imaging element 113, that is, a CMOS image sensor, a CCD, or the like. The illumination unit 111 projects illumination light such as white light toward an imaging field of the imaging element 113, thereby illuminating the subject within the imaging field through the dome-shaped casing 102. The optical system 112 collects reflected light from the imaging field onto an imaging surface of the imaging element 113 and forms an image. The imaging element 113 converts reflected light (optical signal) from the imaging field, received on the imaging surface, into an electrical signal, and outputs it as an image signal.

The control unit 12 operates the imaging unit 11 with a predetermined imaging frame rate, and together with this, allows the illumination unit 111 to project light in synchronization with the imaging frame rate. Moreover, the control unit 12 performs A/D conversion or other predetermined signal processing on the imaging signal generated by the imaging unit 11, thereby generating image data. The control unit 12 further generates an alternating magnetic field from the magnetic field generation unit 14 by allowing the power supply unit 15 to supply power to the magnetic field generation unit 14.

The transmitting unit 13 includes a transmitting antenna, obtains image data signal-processed by the control unit 12 and related information, then performs modulation processing on the data and information, and wirelessly transmits the data and information in sequence to the outside via the transmitting antenna.

The magnetic field generation unit 14 constitutes a portion of a resonant circuit and includes a magnetic field generation coil 141 that generates a magnetic field by the current flow, and a capacitor 142 that forms the resonant circuit together with the magnetic field generation coil 141. The magnetic field generation unit 14 receives power supplied from the power supply unit 15 and generates an alternating magnetic field having a predetermined frequency. The magnetic field generation coil 141 is a cylindrical coil formed by winding metal wire in a fixed direction.

The power supply unit 15 is a power storage unit such as a button cell battery and a capacitor, including a switching unit such as a magnetic switch and an optical switch. When configured to include the magnetic switch, the power supply unit 15 switches power supply on/off by the magnetic field applied from the outside, and in a case of on state, appropriately supplies power of the power storage unit to each of the components (the imaging unit 11, the control unit 12, and the transmitting unit 13) of the capsule endoscope 10. In the case of off state, the power supply unit 15 stops power supply to each of the components of the capsule endoscope 10.

Referring back to FIG. 1, the magnetic field detection apparatus 30 includes a planar panel 31 and a plurality of detection coils 32 arranged on a main surface of the panel 31, each of the detections coils 32 receiving the alternating magnetic field generated from the capsule endoscope 10 and outputting a detected signal.

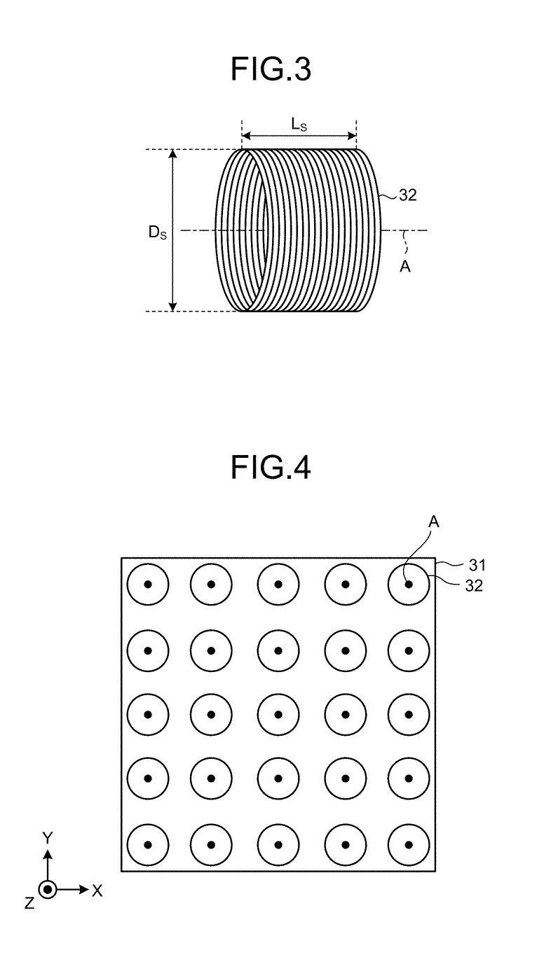

FIG. 3 is a schematic diagram illustrating the shape of each of the detection coils 32. Each of the detection coils 32 is formed by winding meal wire in a fixed direction and has a cylindrical shape in general, as illustrated in FIG. 3. Hereinafter, the diameter of the cylindrical detection coil 32 (cylinder diameter) is defined as Dₛ, the length in the winding direction (cylinder height) is defined as Lₛ, and the ratio of the length Lₛ to the diameter Dₛ, namely, Lₛ/Dₛ, is defined as a parameter indicating a shape of the detection coil 32.

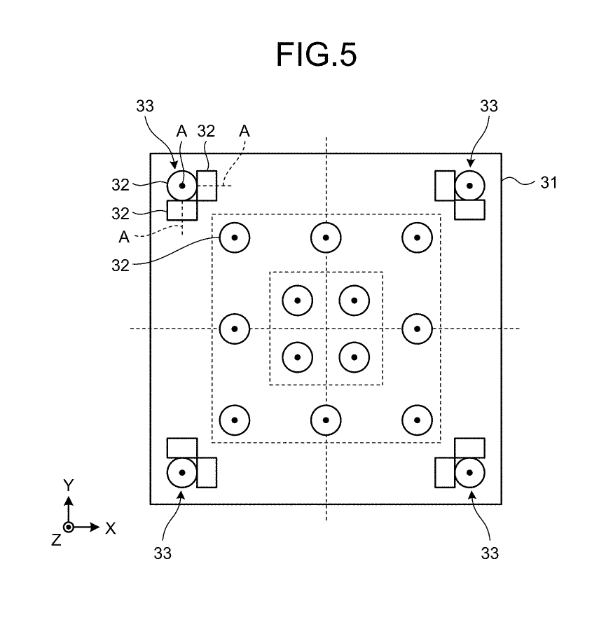

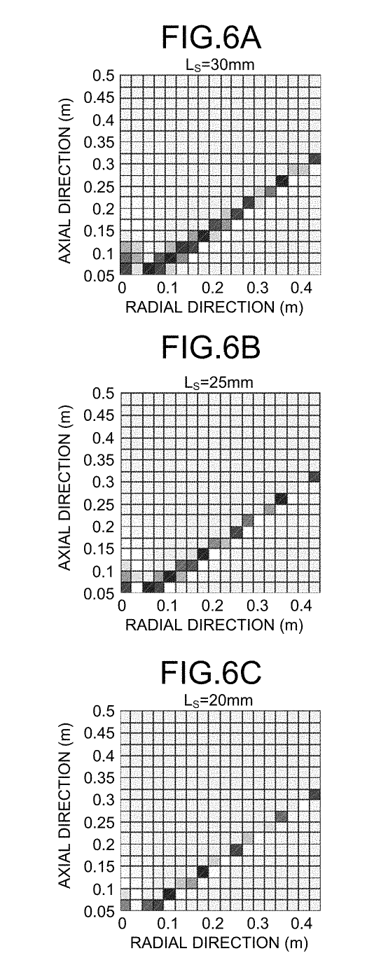

FIGS. 4 and 5 are plan views illustrating exemplary arrangement of the detection coil 32 on the panel 31. The detection coil 32 may be arranged in a matrix in which adjacent detection coils 32 have a uniform interval between each other, as illustrated in FIG. 4. Alternatively, the detection coil 32 may be arranged such that the adjacent detection coils 32 have greater intervals between each other in accordance with a distance from the center of the panel 31, as illustrated in FIG. 5. Moreover, the detection coil 32 may be arranged in the direction in which a rotation center axis A (refer to FIG. 3) is located in parallel with the Z-axis in each of all the detection coils 32 as illustrated in FIG. 4. Alternatively, the direction of the detection coil 32 may be changed so as to allow the rotation center axis A to be in parallel with any of the X-axis, Y-axis, and Z-axis in accordance with the position of the detection coil 32. The detection coil 32 is capable of detecting, with high accuracy, the change in the magnetic field in the direction parallel with the rotation center axis A. Accordingly, by arranging three detection coils 32 in which each of the rotation center axes A is arranged in parallel with each of the X-axis, Y-axis, and Z-axis, as one unit (coil set 33), it is possible to three-dimensionally detect the change in the magnetic field at the corresponding position. FIG. 5 illustrates an exemplary case where a plurality of detection coils 32 is arranged on the inner peripheral side of the panel 31 such that the rotation center axis A is in parallel with the Z-axis, and each of coil sets 33 is arranged at each of ends of the panel 31.

The above-configured magnetic field detection apparatus 30 is arranged in the vicinity of the subject 2 under examination. In the first embodiment, the magnetic field detection apparatus 30 is arranged below the bed 2a such that a main surface of the panel 31 is arranged horizontally.

A region in which the position or the direction of the capsule endoscope 10 can be detected by the magnetic field detection apparatus 30 is defined as a detection target region R. The detection target region R is three-dimensional closed region including a range in which the capsule endoscope 10 is movable within the subject 2 (that is, a range of observation target organ). The detection target region R is preset in accordance with conditions such as the arrangement of the plurality of detections coils 32 on the magnetic field detection apparatus 30 and with magnetic field intensity that can be generated by the magnetic field generation unit 14 within the capsule endoscope 10.

Referring back to FIG. 1, the control apparatus 40 includes a receiving unit 41, an output unit 42, a storage unit 43, a signal processing unit 44, and a calculation unit 45. The receiving unit 41 receives a radio signal transmitted from the capsule endoscope 10 via a receiving antenna 41a. The output unit 42 outputs and displays various types of information, or the like, processed by the control apparatus 40, onto a display device, or the like. The signal processing unit 44 performs various types of signal processing onto detected signals output from each of the detection coils 32 and generates magnetic field information. The calculation unit 45 performs image generation on the basis of image data received by the receiving unit 41 or performs various types of calculation processing including position or direction detection of the capsule endoscope 10 on the basis of the magnetic field information generated by the signal processing unit 44.

When examination is performed with the capsule endoscope 10, a plurality of the receiving antennas 41a is attached on the body surface of the subject 2. Each of the receiving antennas 41a receives radio signals transmitted from the capsule endoscope 10. The receiving unit 41 selects the receiving antenna 41a having the highest reception intensity toward radio signals, among these receiving antennas 41a, and performs demodulation processing, or the like, onto the radio signals received via the selected receiving antenna 41a, thereby obtaining image data of in-vivo images and related information.

The output unit 42 includes various displays such as liquid crystal display and an organic EL display, and displays in-vivo image of the subject 2 and information on the position and direction of the capsule endoscope 10 when the in-vivo image is captured.

The storage unit 43 is configured with a storage medium and a read/write apparatus for rewritably storing information, such as a flash memory and a hard disk. The storage unit 43 stores various programs, parameters used for controlling components of the control apparatus 40 by the calculation unit 45, image data of the in-vivo image captured by the capsule endoscope 10, and information on the position and direction of the capsule endoscope 10 within the subject 2, or the like.

The signal processing unit 44 includes a filter unit 441, an amplifier 442, and an A/D converter 443. The filter unit 441 shapes the waveform of the detected signal output from the magnetic field detection apparatus 30. The A/D converter 443 performs A/D conversion processing on the detected signal.

The calculation unit 45 is formed with a central processing unit (CPU), for example, integrally controls operation of the control apparatus 40, specifically, by reading a program from the storage unit 43, transferring instructions and data to each of components constituting the control apparatus 40, or performing other operation. The calculation unit 45 further includes an image processing unit 451 and a position detection calculation unit 452.

The image processing unit 451 performs predetermined image processing such as white balance processing, demosaicing, gamma conversion, smoothing (noise removal, etc.) toward the image data input from the receiving unit 41, and thereby generating image data for display.

The position detection calculation unit 452 obtains information representing the position and direction of the capsule endoscope 10 (hereinafter, collectively referred to as positional information) on the basis of the detected signal output from the signal processing unit 44. More specifically, the position detection calculation unit 452 includes an FFT processing unit 452a and a position calculation unit 452b. The FFT processing unit 452a extracts magnification field information such as amplitude and phase of the alternating magnetic field by performing fast Fourier transform (hereinafter, referred to as FFT processing) on the detection data output from the signal processing unit 44. The position calculation unit 452b calculates at least any of the position and the direction of the capsule endoscope 10 on the basis of the magnetic field information extracted by the FFT processing unit 452a.

Nest, the shape of the detection coil 32 disposed on the magnetic field detection apparatus 30 will be described. In many cases, position detection errors for the capsule endoscope 10 is attributable to an error arising between distribution of a theoretical magnetic field (hereinafter, referred to as an ideal magnetic field) when the position of the magnetic field generation coil 141 is assumed to be a magnetic field generation source, and distribution of a magnetic field based on the actual magnetic field (hereinafter, referred to as a detected magnetic field) actually detected by the plurality of detection coils 32. This error arises from the fact that the position calculation unit 452b calculates the position or the direction using magnetic field distribution in an ideal condition in which the magnetic field generation coil 141 and the detection coil 32 are assumed as points, without taking the size and shape of the magnetic field generation coil 141 and the detection coil 32 into consideration.

Accordingly, the present inventor has simulated to determine the error (detected magnetic field error) between the intensity of the detected magnetic field and the intensity of the ideal magnetic field in the following procedure. Specifically, it is assumed that the detection coil 32 is arranged at one position within an arrangement surface of the detection coil 32, and a center point (geometrical center point) in each of the radial direction and the length direction of the detection coil 32 is defined as an origin (X, Y, Z)=(0, 0, 0). Subsequently, a magnetic field intensity detected by the detection coil 32 at the time when the magnetic field generation coil 141 is arranged at a predetermined measurement point within the detection target region, is calculated. The magnetic field intensity in a case where the magnetic field generation coil 141 and the detection coil 32 are assumed as points (microscopic points) is defined as the ideal magnetic field intensity, and magnetic field intensity in a case where the magnetic field generation coil 141 and the detection coil 32 have their actual sizes and shapes is defined as the detected magnetic field intensity. Subsequently, the detected magnetic field error was calculated from a difference between the above-described detected magnetic field intensity and the ideal magnetic field intensity (detected magnetic field intensity−ideal magnetic field intensity).

The ideal magnetic field intensity was obtained by calculating magnetic field distribution on the assumption that a microscopic magnetic power generation source exists at each of the measurement points.

Meanwhile, the following model was set for the detected magnetic field intensity. The position is set such that the centers in the radial direction and in the length direction coincide with the origin, and the magnetic field intensity on the origin is obtained on the assumption that the detection coil 32 is a set of circular detection coils having a diameter Dₛ aggregated across a length Lₛ, without taking the helical shape of the wound metal wire into consideration. The direction of the detection coil 32 is determined such that the rotation center axis of the detection coil 32 is vertical (parallel to the Z-axis, namely, opening end surface is horizontal). The shape of the detection coil 32 was determined such that the diameter Dₛ was set to four types of 10 mm, 20 mm, 30 mm, and 40 mm, and the length Lₛ was varied in a range 5 mm to 30 mm with respect to each of the diameters.

The position is set such that the center points in the radial direction and in the length direction coincide with coordinates of each of the measurement points, and the magnetic field distribution is calculated on the assumption that the magnetic field generation coil 141 is a set of circular current having a diameter Dₘ aggregated across a length Lₘ, without taking the helical shape of the wound metal wire into consideration. As the direction of the magnetic field generation coil 141 at each of the measurement points, two patterns were applied, namely, the direction in which the rotation center axis of the magnetic field generation coil 141 is vertical (that is, the same as the direction of the detection coil 32) and the direction in which the rotation center axis is parallel to the X-axis (that is, radial direction of the detection coil). The diameter Dₘ of the magnetic field generation coil 141 is determined to be smaller than any of the above-described diameters Dₛ of the detection coil 32.

The measurement points to be set within the detection target region were determined to be arranged with a pitch of 50 mm in the range of 0 mm to 450 mm in the +X direction, and with a pitch of 50 mm in the range of 50 mm to 500 mm in the +Z direction. Note that the arrangement in the −X direction and the ±Y directions is symmetrical to the +X direction in relation with arrangement of the detection coil 32, and thus, the simulation therefor was omitted.

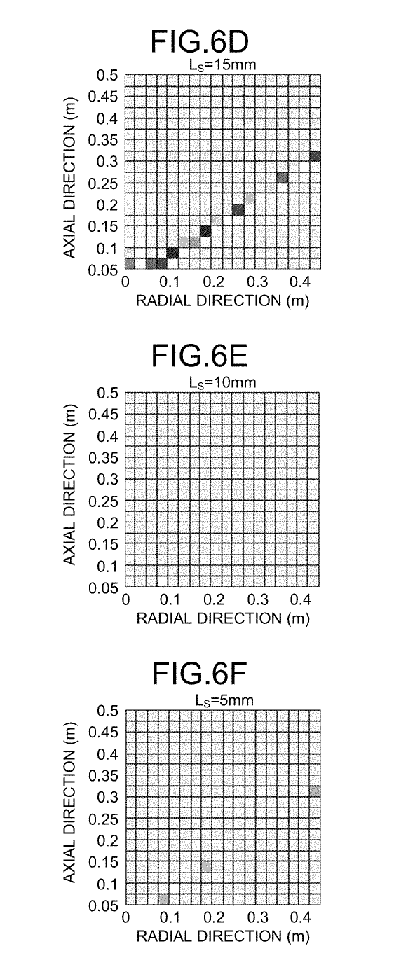

FIGS. 6A to 6F are schematic diagrams illustrating results of the above-described simulation. In this exemplary case, the diameter Dₛ of the detection coil 32 was 10 mm. The length Lₛ of the detection coil 32 is 30 mm (Lₛ/Dₛ=3.0) in the case of FIG. 6A, 25 mm (Lₛ/Dₛ=2.5) in the case of FIG. 6B, 20 mm (Lₛ/Dₛ=2.0) in the case of FIG. 6C, 15 mm (Lₛ/Dₛ=1.5) in the case of FIG. 6D, 10 mm (Lₛ/Dₛ=1.0) in the case of FIG. 6E, and 5 mm (Lₛ/Dₛ=0.5) in the case of FIG. 6F.

In the graphs illustrated in FIGS. 6A to 6F, the horizontal axis indicates the coordinates in the radial direction (X-direction) of the detection coil 32, and the vertical axis indicates the coordinates in the axial direction (Z-direction) of the detection coil 32. The density of each of the coordinate points within the graph indicates an absolute value of the detected magnetic field error. Specifically, the density indicates such that the higher the density on the coordinate, the greater the detected magnetic field error (absolute value), and the lower the density, the smaller the detected magnetic field error (absolute value).

The present inventor has found the following as a result of the simulation. When comparing FIGS. 6A to 6F with each other, it is understood that the detected magnetic field error is properly suppressed (there are few high-density regions) in the case of Lₛ/Dₛ=1.0 illustrated in FIG. 6E. In other words, it would be appropriate to determine that the closer the diameter Dₛ to the length Lₛ of the detection coil 32, the smaller the detected magnetic field error.

Moreover, in a case where the same measurement points are used (that is, the ideal magnetic field intensity is the same), it was found that the greater the diameter Dₛ of the detection coil 32, the smaller the detected magnetic field intensity. Therefore in this case, the difference from the ideal magnetic field intensity shifts in the negative direction. Meanwhile, in a case where the same measurement points are used (same as above), it was found that the longer the length Lₛ of the detection coil, the greater the detected magnetic field intensity. Therefore in this case, the difference from the ideal magnetic field intensity shifts in the positive direction. The present inventor thought, from these result, that it would be possible to reduce the detected magnetic field error by adjusting the balance between the diameter Dₛ and the length Lₛ of the detection coil 32, and performed further examination in order to obtain the optimum shape for the detection coil 32.

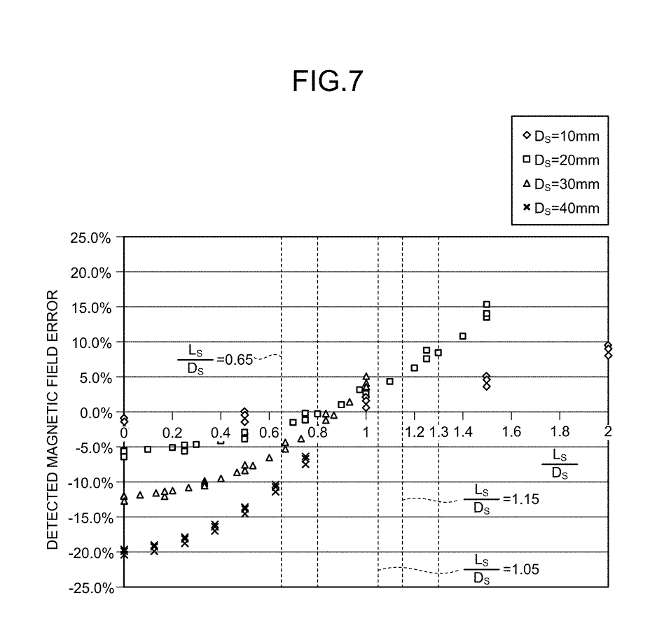

FIG. 7 is a summary of the simulation related to the above-described four types of detection coils 32 (Dₛ=10 mm, 20 mm, 30 mm, and 40 mm), representing the correlation between the ratio of the length Lₛ to the diameter Dₛ, namely, Lₛ/Dₛ (horizontal axis) and the detected magnetic field error (vertical axis).

As illustrated in FIG. 7, it is observed that, in order to control the detected magnetic field error to ±20% or below, it would be appropriate to set the ratio Lₛ/Dₛ, that is, the ratio of the length to the diameter, of the detection coil 32, to a value greater than 0 and not greater than 1.3. Furthermore, in order to control the detected magnetic field error to ±10% or below, it would be appropriate to set the ratio Lₛ/Dₛ, that is, the ratio of the length to the diameter, to a value being 0.65 or above and 1.15 or below. Furthermore, in order to control the detected magnetic field error to ±5% or below, it would be appropriate to set the ratio Lₛ/Dₛ, that is, the ratio of the length to the diameter, to a value being 0.8 or above and 1.05 or below. Herein, the detected magnetic field error being ±20% represents a range whereby the error (position detection error) between the position of the capsule endoscope 10 based on the magnetic field detected by the detection coil 32 and the actual position of the capsule endoscope 10 is 2 mm or below. Moreover, the detected magnetic field error being ±10% represents a range whereby the position detection error is 1 mm or below.

The present inventor further found, from the above-described simulation, that one can calculate approximately the detected magnetic field error by using the ratio Lₛ/Dₛ, that is, the ratio of the length Lₛ to the diameter Dₛ, of the detection coil 32. The following Formula (1) is approximation representing a detected magnetic field error B.

$$B = \vert {G}_{1} {( \frac{{L}_{S}}{{D}_{S}} )}^{2} + {G}_{2} ( \frac{{L}_{S}}{{D}_{S}} ) + {G}_{3} \vert \qquad ( 1 )$$

Coefficients G₁, G₂, and G₃ illustrated in Formula (1) change in accordance with the diameter Dₛ of the detection coil 32. Each of the following Formulae (2) to (4) is approximation that respectively gives each of the coefficients G₁, G₂, and G₃.

G₁=−1.73×10⁻⁵×Dₛ²+7.36×10⁻³×Dₛ−4.71×10⁻² (2)

G₂=3.74×10⁻⁵×Dₛ²−1.54×10⁻³×Dₛ+1.16×10⁻² (3)

G₃=−8.96×10⁻⁵×Dₛ²−1.74×10⁻³×Dₛ+1.30×10⁻² (4)

Accordingly, in order to achieve the detected magnetic field error B that is a desired value or below, it would be sufficient to obtain the ratio Lₛ/Dₛ that satisfies the following Formula (5).

$$\vert {G}_{1} {( \frac{{L}_{S}}{{D}_{S}} )}^{2} + {G}_{2} ( \frac{{L}_{S}}{{D}_{S}} ) + {G}_{3} \vert ≦ B \qquad ( 5 )$$

In a case where it is desired to achieve the detected magnetic field error of 10% or below, for example, and when the diameter Dₛ of the detection coil 32 is fixed, it would be possible to obtain the length Lₛ of the detection coil 32, capable of achieving the detected magnetic field error of 10% or below, by solving Formula (5) while applying B=0.1 onto the right side of Formula (5). In contrast, when the length Lₛ of the detection coil 32 is fixed, it would be possible to obtain the diameter Dₛ of the detection coil 32, capable of achieving the detected magnetic field error of 10% or below, by solving Formula (5) while entering B=0.1 on the right side of Formula (5).

As described above, according to the first embodiment of the disclosure, by setting the ratio Lₛ/Dₛ, that is, the ratio of the length Lₛ to the diameter Dₛ, of the detection coil 32, to a value greater than 0 and not greater than 1.3, preferably, 0.65 or above and 1.15 or less, further preferably, 0.8 or above and 1.05 or less, it is possible to sufficiently and stably reduce the detected magnetic field error at the position of the detection coil 32. Accordingly, by using above-designed the detection coil 32, it is possible to execute position detection of the capsule endoscope 10 with high accuracy.

### Second Embodiment

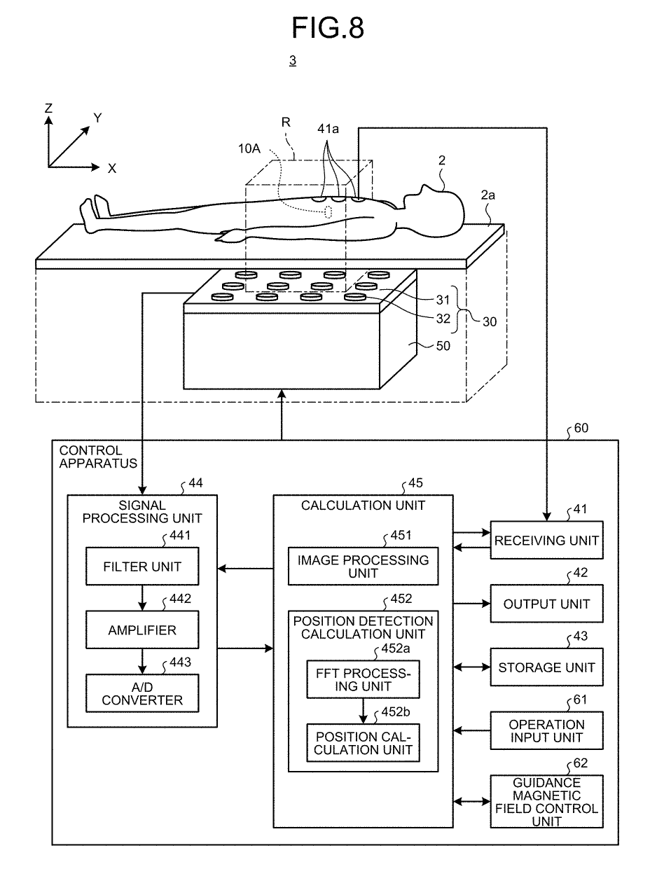

Next, a second embodiment of the disclosure will be described. FIG. 8 is a schematic diagram illustrating an exemplary configuration of a capsule medical apparatus guidance system according to the second embodiment of the disclosure. As illustrated in FIG. 8, a capsule medical apparatus guidance system 3 according to the second embodiment includes a capsule endoscope 10A, the magnetic field detection apparatus 30, a guidance magnetic field generation apparatus 50, and a control apparatus 60. The guidance magnetic field generation apparatus 50 generates a magnetic field for guiding the capsule endoscope 10A. The control apparatus 60 detects the position or direction of the capsule endoscope 10A and controls operation of the guidance magnetic field generation apparatus 50. Among these, the configuration of the magnetic field detection apparatus 30 is similar to the configuration in the first embodiment.

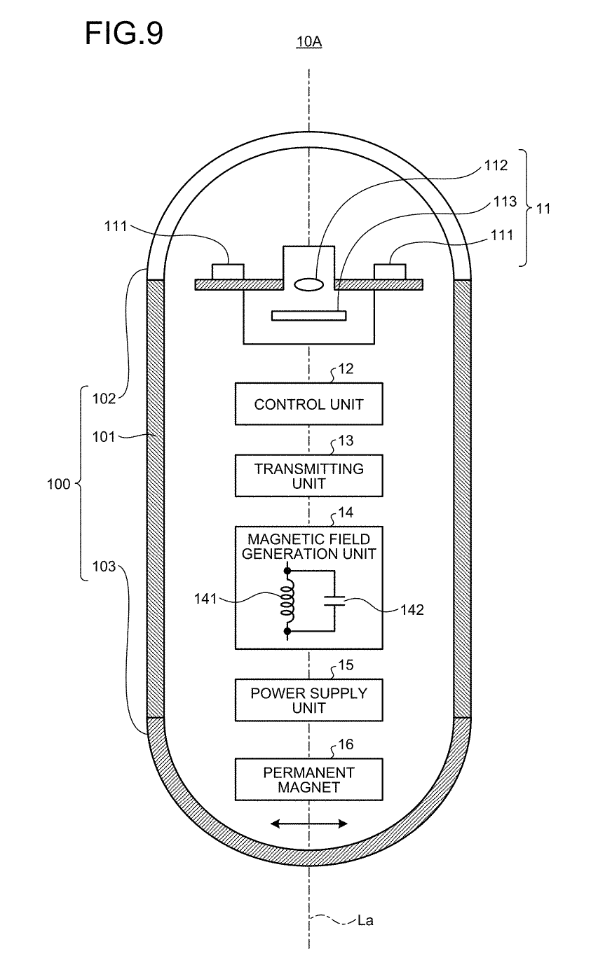

FIG. 9 is a schematic diagram illustrating an exemplary internal structure of the capsule endoscope 10A. As illustrated in FIG. 9, the capsule endoscope 10A further includes a permanent magnet 16, in addition to the capsule endoscope 10 illustrated in FIG. 2. Configurations and operation of each of the components of the capsule endoscope 10A other than the permanent magnet 16 are similar to the case in the first embodiment.

The permanent magnet 16 is provided to enable magnetic guidance of the capsule endoscope 10A by the magnetic field generated by the guidance magnetic field generation apparatus 50. The permanent magnet 16 is fixed inside the casing 100 such that the magnetization direction has inclination toward a long axis La of the casing 100. Note that in FIG. 9, the magnetization direction of the permanent magnet 16 is indicated with an arrow. In the second embodiment, the permanent magnet 16 is arranged such that the magnetization direction is orthogonal to the long axis La. The permanent magnet 16 operates to follow the magnetic field applied from the outside, making it possible to achieve magnetic guidance of the capsule endoscope 10A by the guidance magnetic field generation apparatus 50.

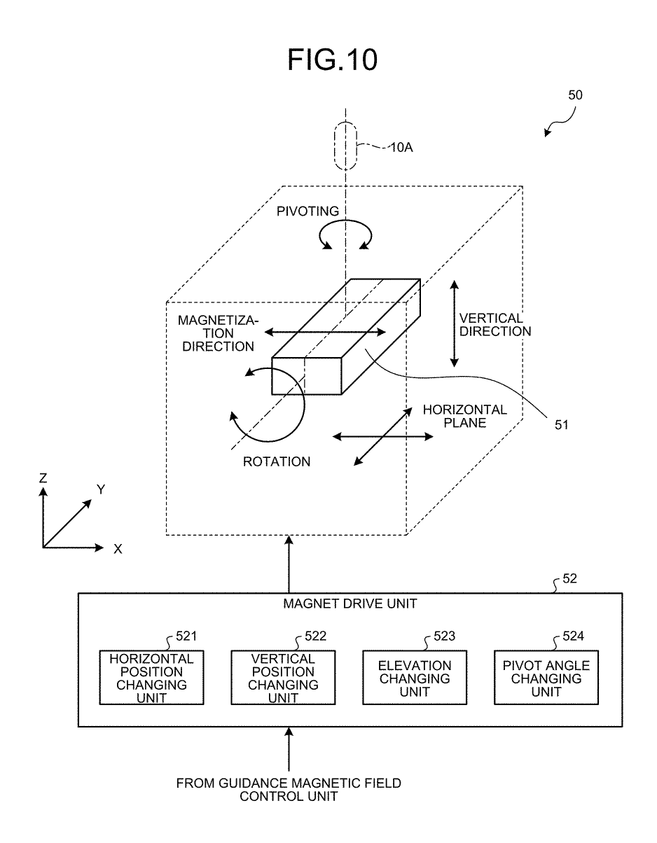

FIG. 10 is a schematic diagram illustrating an exemplary configuration of the guidance magnetic field generation apparatus 50. As illustrated in FIG. 10, the guidance magnetic field generation apparatus 50 generates a magnetic field for changing, relative to the subject 2, the position, an inclination angle of the long axis La with respect to the vertical direction, and azimuth, of the capsule endoscope 10A introduced into the subject 2. More specifically, the guidance magnetic field generation apparatus 50 includes an external permanent magnet 51 and a magnet drive unit 52. The external permanent magnet 51 functions as a guidance magnetic field generation unit. The magnet drive unit 52 changes the position and posture of the external permanent magnet 51. Among these, the magnet drive unit 52 includes a horizontal position changing unit 521, a vertical position changing unit 522, an elevation changing unit 523, and a pivot angle changing unit 524.

The external permanent magnet 51 is preferably formed with a bar magnet having a rectangular parallelepiped shape, and contains the capsule endoscope 10A within a region formed by one of four planes parallel to the magnetization direction of the magnet, being projected to the horizontal plane. Note that it is allowable to provide an electromagnet that generates a magnetic field by current flow, instead of the external permanent magnet 51.

The magnet drive unit 52 operates in accordance with a control signal output from a guidance magnetic field control unit 62 described below. Specifically, the horizontal position changing unit 521 translates the external permanent magnet 51 within the X-Y plane. That is, the external permanent magnet 51 is moved within the horizontal plane while the relative positions of two magnetic poles magnetized on the external permanent magnet 51 being maintained. The vertical position changing unit 522 translates the external permanent magnet 51 along the Z-direction. That is, the external permanent magnet 51 is moved along the vertical direction while the relative positions of two magnetic poles magnetized on the external permanent magnet 51 being maintained. The elevation changing unit 523 changes the magnetization direction angle with respect to the horizontal plane by rotating the external permanent magnet 51 within the vertical plane including the magnetization direction, on the external permanent magnet 51. The pivot angle changing unit 524 pivots the external permanent magnet 51 around a vertical direction axis passing through the center of the external permanent magnet 51.

Referring back to FIG. 8, the control apparatus 60 further includes an operation input unit 61 and a guidance magnetic field control unit 62, in addition to the control apparatus 40 illustrated in FIG. 1. Configurations and operation of each of the components of the control apparatus 60 other than the operation input unit 61 and the guidance magnetic field control unit 62 are similar to the case in the first embodiment.

The operation input unit 61 is configured with input devices such as various buttons, a switch, and a keyboard, pointing devices such as a mouse and a touch panel, and a joystick, or the like. The operation input unit 61 is used to input various types of information and commands into the control apparatus 60. The operation input unit 61 inputs various types of information into the calculation unit 45 in response to input operation by the user. The information input by the operation input unit 61 includes, for example, information for guiding the capsule endoscope 10A to the position and posture desired by the user (hereinafter, referred to as guidance operation information).

The guidance magnetic field control unit 62 performs control for guiding the capsule endoscope 10A. Specifically, in a case where guidance operation information is input from the operation input unit 61, the guidance magnetic field control unit 62 controls operation of each of components of the magnet drive unit 52 such that the capsule endoscope 10A is arranged in the position and direction desired by the user, on the basis of the guidance operation information and the position and direction of the capsule endoscope 10A calculated by the position detection calculation unit 452. In other words, the magnetic field in space, including the position of the capsule endoscope 10A is changed by changing the position, elevation, and the pivot angle of the external permanent magnet 51, thereby guiding the capsule endoscope 10A.

The first and second embodiments of the disclosure have been described hereinabove merely as examples for implementation of the disclosure, and thus, the disclosure is not intended to be limited to these embodiments. Furthermore, the disclosure can be modified in various manners in accordance with the specification, or the like, and it is apparent from the description given above that various other modes can be implemented within the scope of the disclosure.

According to some embodiments, the magnetic field generated by the magnetic field generation unit is detected by a plurality of detection coils each having the ratio of the length to the diameter being more than zero and not more than 1.3. Accordingly, it is possible to sufficiently and stably reduce a detected magnetic field error on each of the detection coils.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A position detection system comprising:
a cylindrical detection coil configured to detect a magnetic field generated by a magnetic field generation unit; and
a calculation unit configured to calculate at least one of a position and a direction of the magnetic field generation unit based on the magnetic field detected by the detection coil,
wherein a relationship between a diameter Ds and a length Ls in a winding direction, of the detection coil, satisfies Formula (1), and
each of coefficients G₁, G₂, and G₃ in Formula (1) is respectively given by each of Formulae (2), (3) and (4).
$$\begin{matrix}\vert {G}_{1} {( \frac{{L}_{S}}{{D}_{S}} )}^{2} + {G}_{2} ( \frac{{L}_{S}}{{D}_{S}} ) + {G}_{3} \vert ≦ 0.2 & ( 1 ) \\ {G}_{1} = - 1.73 {10}^{- 5} {D}_{S}^{2} + 7.36 {10}^{- 3} {D}_{S} - 4.71 {10}^{- 2} & ( 2 ) \\ {G}_{2} = 3.74 {10}^{- 5} {D}_{S}^{2} - 1.54 {10}^{- 3} {D}_{S} + 1.16 {10}^{- 2} & ( 3 ) \\ {G}_{3} = - 8.96 {10}^{- 5} {D}_{S}^{2} - 1.74 {10}^{- 3} {D}_{S} + 1.30 {10}^{- 2} & ( 4 )\end{matrix}$$

2. The position detection system according to claim 1, further comprising the magnetic field generation unit,
wherein the magnetic field generation unit includes a magnetic field generation coil formed in a cylindrical shape and configured to generate a magnetic field, and
the diameter of the detection coil is greater than a diameter of the magnetic field generation coil.

3. The position detection system according to claim 2, further comprising a capsule medical apparatus incorporating the magnetic field generation unit.

4. A capsule medical apparatus guidance system comprising the position detection system according to claim 3,
the capsule medical apparatus further incorporating a magnet, the capsule medical apparatus guidance system further comprising a guidance magnetic field generation apparatus configured to generate a magnetic field for guiding the capsule medical apparatus by causing the magnet to act.
